(19) United States
(12) Patent Application Publication
(10) Pub. No.: US 2025/0297930 A1
(43) Pub. Date: Sep. 25, 2025

(54) SAMPLE PROCESSING METHOD AND SEMICONDUCTOR DEVICE ANALYSIS METHOD INCLUDING THE SAME

(71) Applicant: SAMSUNG ELECTRONICS CO., LTD., Suwon-si (KR)

(72) Inventors: Minchul JO, Suwon-si (KR); Sanghyun PARK, Suwon-si (KR); Kyusang CHO, Suwon-si (KR); Gilho GU, Suwon-si (KR)

(73) Assignee: SAMSUNG ELECTRONICS CO., LTD., Suwon-si (KR)

(21) Appl. No.: 18/883,620

(22) Filed: Sep. 12, 2024

(30) Foreign Application Priority Data
Mar. 22, 2024 (KR) 10-2024-0039924

(51) Int. Cl. G01N 1/28; G01N 1/44; G01N 23/2251; G01N 33/00

(52) U.S. Cl. CPC G01N 1/286; G01N 1/44; G01N 23/2251; G01N 33/0095; G01N 2001/2873

(57) ABSTRACT

Sample processing methods and semiconductor device analysis methods are provided. A sample processing method includes: preparing a sample that has a first surface and a second surface that are opposite to each other; forming a guide line that extends in a first direction by irradiating the first surface of the sample with a laser, and destroying the sample along the guide line, wherein the sample includes a substrate that has a bottom surface that extends parallel to the first direction, the bottom surface of the substrate has a (100) crystal plane, and the laser includes a femtosecond pulse laser or a nanosecond pulse laser.

### CROSS-REFERENCE TO RELATED APPLICATION

This U.S. non-provisional application claims priority under 35 U.S.C § 119 to Korean Patent Application No. 10-2024-0039924, filed on Mar. 22, 2024, in the Korean Intellectual Property Office, the disclosure of which is hereby incorporated by reference in its entirety.

### BACKGROUND

### 1. Field

Embodiments of the present disclosure relate to a sample processing method and a semiconductor device analysis method including the same, and more particularly, to a sample processing method using a laser and a semiconductor device analysis method including the same.

### 2. Brief Description of Related Art

The continuing integration of semiconductor devices causes complexity of layers included in semiconductor devices and internal connection lines in semiconductor devices. Since internal defects in semiconductor devices are detrimental to reliability and performance of products, technology for inspecting these defects is important. In particular, when structures are observed in a vertical direction, an increase in aspect ratio due to integration induces an increase in the difficulty of required sample processing.

### SUMMARY

Some embodiments of the present disclosure provide a sample processing method for a substrate having a (100) crystal plane and a semiconductor device analysis method including the same.

Some embodiments of the present disclosure provide a sample processing method which is fast and cost-effective while minimizing effects on semiconductor devices and a semiconductor device analysis method including the same.

According to embodiments of the present disclosure, a sample processing method is provided and includes: preparing a sample that has a first surface and a second surface that are opposite to each other; forming a guide line that extends in a first direction by irradiating the first surface of the sample with a laser; and destroying the sample along the guide line, wherein the sample includes a substrate that has a bottom surface that extends parallel to the first direction, the bottom surface of the substrate has a (100) crystal plane, and the laser includes a femtosecond pulse laser or a nanosecond pulse laser.

According to embodiments of the present disclosure, a semiconductor device analysis method is provided and includes: processing a wafer, that includes a substrate and a device layer on the substrate, such as to obtain a sample from the wafer; processing the sample obtained from the wafer; and analyzing the sample, wherein processing the sample includes: forming an opening in the substrate by irradiating a top surface of the substrate with a laser; and destroying the sample along the opening, wherein a distance between a bottom surface of the opening and a bottom surface of the substrate is in a range of 80 μm to 120 μm, and wherein the destroying the sample includes cutting the sample in a [100] crystal direction.

According to embodiments of the present disclosure, a non-transitory computer readable medium storing computer instructions is provided. The computer instructions may be configured to, when executed by at least one processor, cause the at least one processor to: prepare a sample that has a first surface and a second surface that are opposite to each other; form a guide line that extends in a first direction by irradiating the first surface of the sample with a laser; and destroy the sample along the guide line, wherein the sample includes a substrate that has a bottom surface that extends parallel to the first direction, the bottom surface of the substrate has a (100) crystal plane, and the laser includes a femtosecond pulse laser or a nanosecond pulse laser.

Aspects of embodiments of the present disclosure are not limited to the aspects mentioned above, and other aspects which have not been mentioned above will be clearly understood to those skilled in the art from the following description.

### BRIEF DESCRIPTION OF DRAWINGS

FIG. 1 illustrates a flow chart showing a semiconductor device analysis method according to some embodiments of the present disclosure.

FIGS. 2 to 9 illustrate diagrams showing a semiconductor device analysis method according to some embodiments of the present disclosure.

### DETAILED DESCRIPTION

The following will now describe some non-limiting example embodiments of the present disclosure with reference to the accompanying drawings. Like reference numerals may indicate like components throughout the description.

It will be understood that when an element or layer is referred to as being “on,” “connected to,” or “coupled to” another element or layer, it can be directly on, connected to, or coupled to the other element or layer or intervening elements or layers may be present. In contrast, when an element or layer is referred to as being “directly on,” “directly connected to,” or “directly coupled to” another element or layer, there are no intervening elements or layers present.

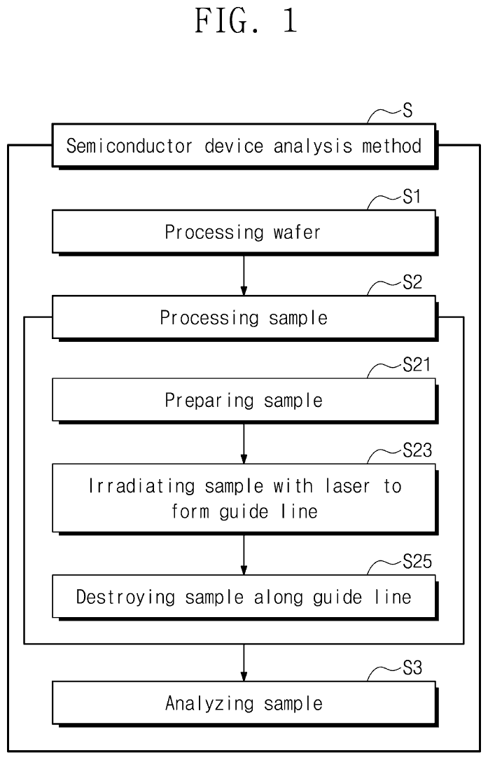

FIG. 1 illustrates a flow chart showing a semiconductor device analysis method according to some embodiments of the present disclosure.

Referring to FIG. 1, a semiconductor device analysis method S may be provided. For example, the semiconductor device analysis method S may be a method of analyzing a sample obtained by processing wafer including a semiconductor device. The semiconductor device analysis method S may include processing a wafer (operation S1), processing a sample (operation S2), and analyzing the sample (operation S3).

The sample process step (operation S2) may include preparing a sample (operation S21). Irradiating the sample with a laser to form a guide line (operation S23), and destroying the sample along the guide line (operation S25).

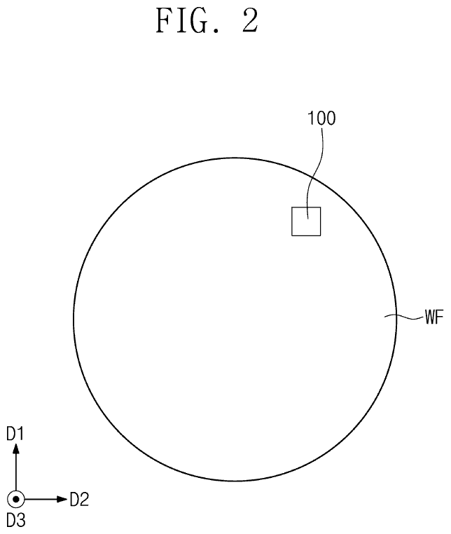

With reference to FIGS. 2 to 9, the following will describe in detail the semiconductor device analysis method S of FIG. 1.

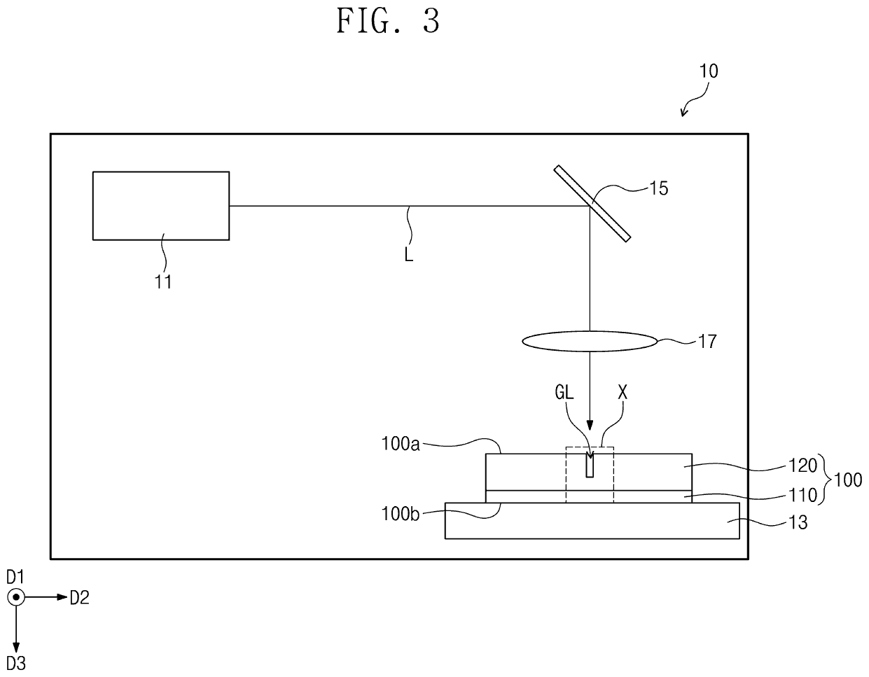

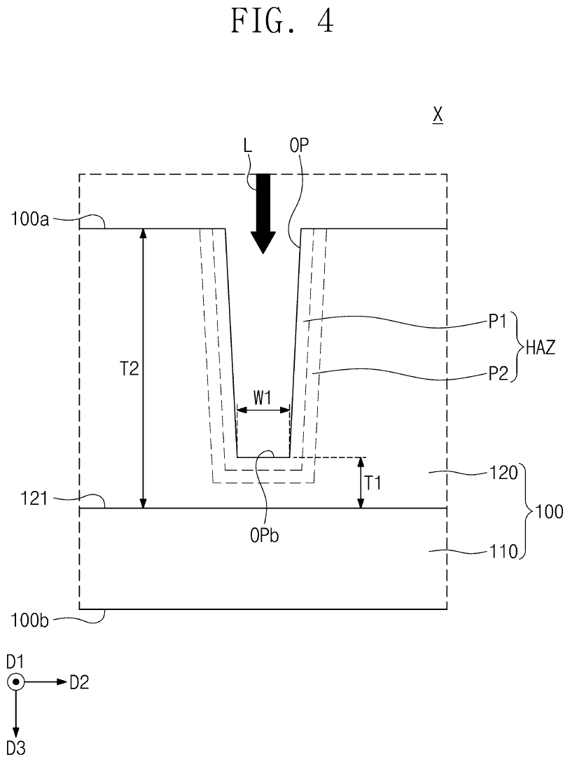

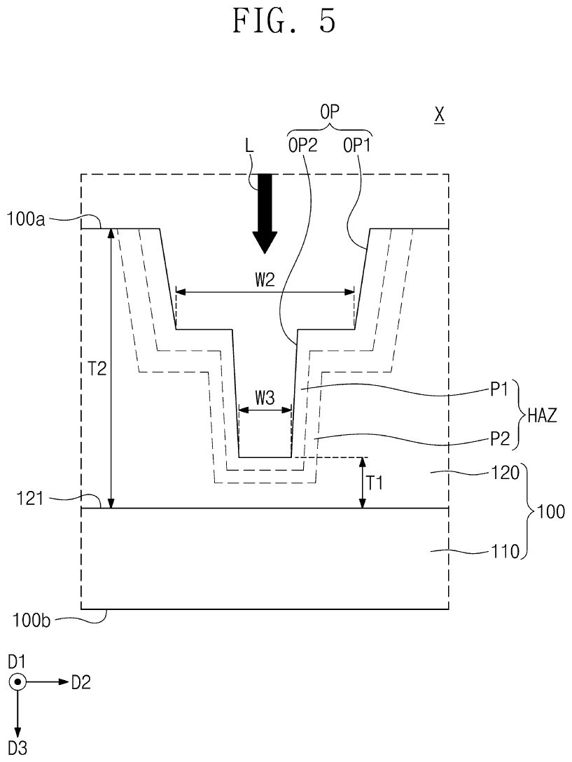

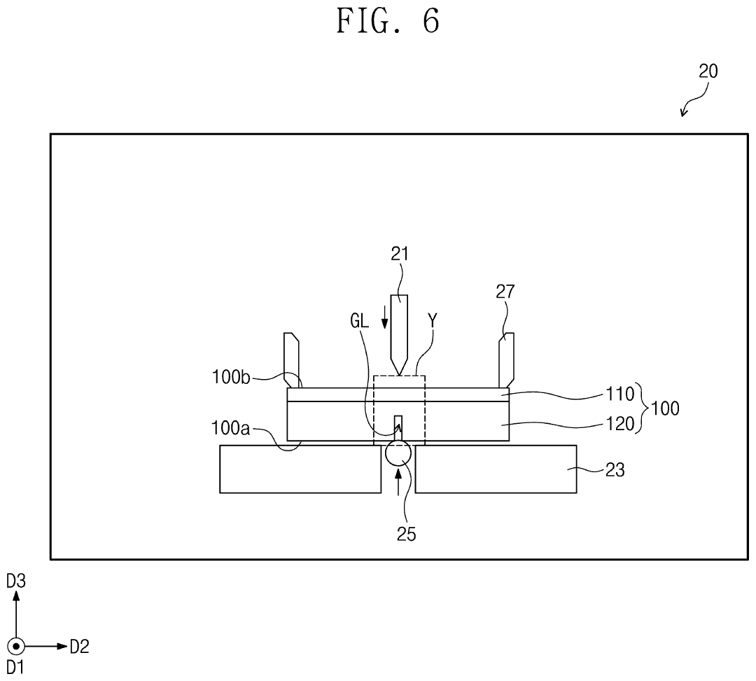

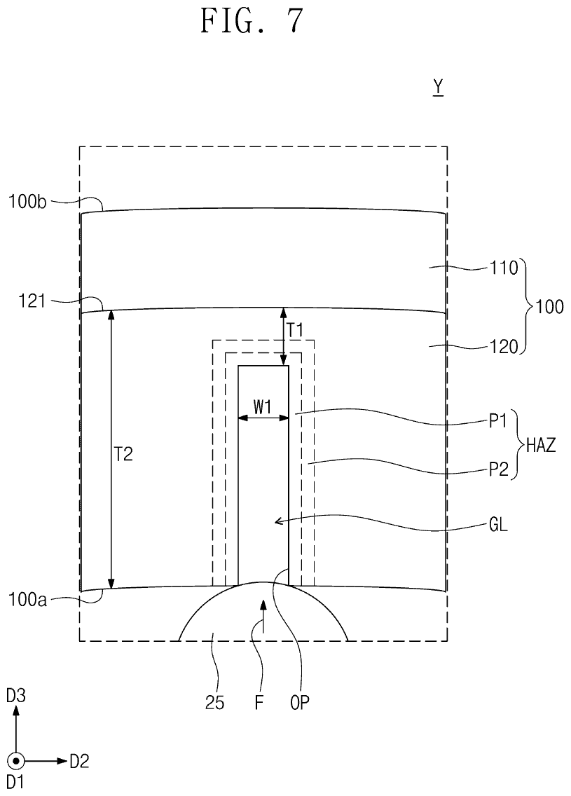

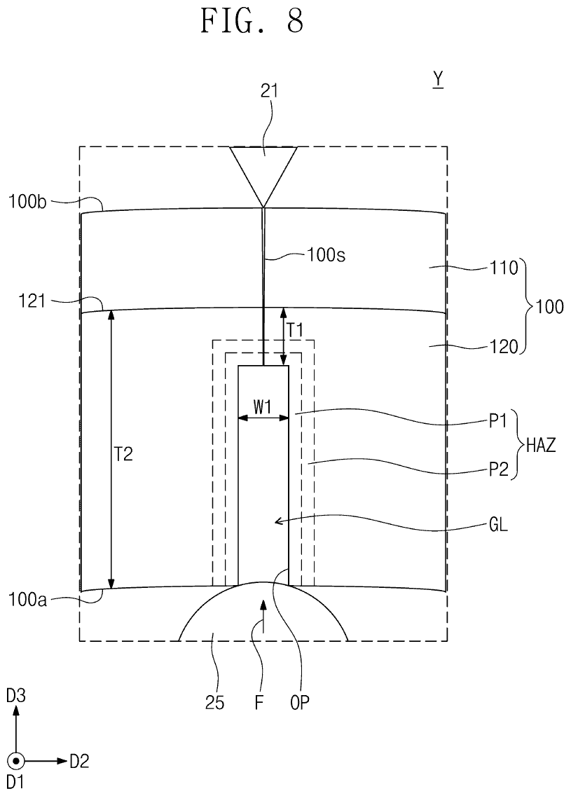

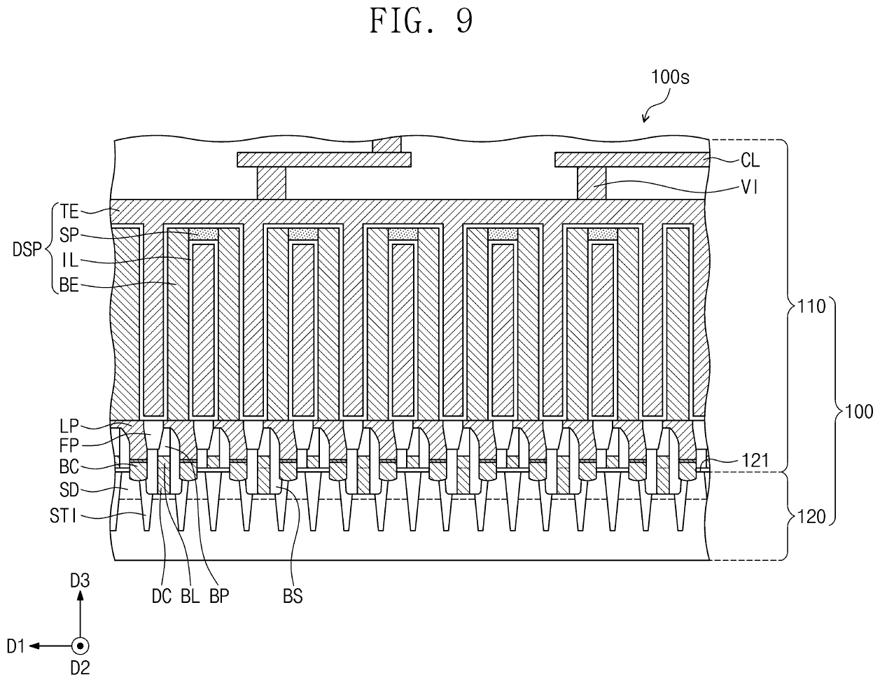

FIGS. 2 to 9 illustrate diagrams showing a semiconductor device analysis method according to some embodiments of the present disclosure. FIGS. 4 and 5 illustrate enlarged views showing section X of FIG. 3. FIGS. 7 and 8 illustrate enlarged views showing section Y of FIG. 6. FIG. 9 illustrates an enlarged view showing a cross-section of a sample according to some embodiments of the present disclosure.

Referring to FIGS. 1 and 2, the wafer process step (operation S1) may include preparing a wafer WF and cutting a portion of the wafer WF to obtain a sample 100. In this description, the wafer WF may be a wafer in which a substrate is provided thereon with a semiconductor device formed by semiconductor fabrication process. For example, the substrate may include a silicon (Si) wafer, a silicon carbide (SiC) wafer, a gallium arsenide (GaAs) wafer, or a monocrystalline silicon wafer. The semiconductor device may include one or more of a static random access memory (SRAM), a dynamic random access memory (DRAM), a NAND Flash memory, and a logic circuit. However, embodiments of present disclosure are not limited thereto.

A dicing process may be performed such that a portion of the wafer WF may be removed to obtain the sample 100. For example, a portion of the wafer WF may be cut through the dicing process. Thus, the sample 100 may be separated from the wafer WF. The dicing process may use a blade, a laser, and/or a plasma, but embodiments of the present disclosure are not limited thereto.

Referring to FIGS. 1 and 3, the sample preparation step (operation S21) may include allowing a laser treatment apparatus 10 to receive the sample 100 separated from the wafer WF of FIG. 2. The laser treatment apparatus 10 may include a laser generator 11, a first stage 13, a reflection mirror 15, and a lens 17.

The laser generator 11 may generate a laser L. For example, the laser generator 11 may generate a femtosecond pulse laser. Since the femtosecond pulse laser has a narrow pulse width of about 10⁻¹⁵ seconds, a pulse time of the laser L may be shorter than a thermal diffusion time caused by the laser L. Thus, there may be a reduction in thermal deformation of the sample 100 to which the laser Lis irradiated. According to some embodiments, the laser generator 11 may generate a nanosecond pulse laser.

For example, the laser L produced from the laser generator 11 may have a laser power of about 10 W to about 50 W, a laser pulse duration of about 500 fs to about 500 ns, a laser pulse repetition rate of about 10 kHz to about 2,000 kHz, a nominal pulse energy of about 50 μJ to about 300 μJ, a laser wavelength of about 300 nm to about 1,100 nm, and a laser scanning speed of about 10 mm/s to about 3,000 mm/s.

In addition, the laser generator 11 may include a solid medium for allowing the laser L to pass therethrough. Properties of the laser L may depend on the solid medium. For example, the solid medium may include one or more of a neodymium-doped yttrium aluminum garnet (Nd:YAG) compound, a neodymium-doped yttrium orthovanadate (Nd:YVO₄) compound, an aluminum gallium arsenide (AlGaAs) compound, an aluminum gallium indium phosphide (AlGaInP) compound, gallium nitride (GaN) compound, neodymium-doped optical fiber (Nd-fiber), and sapphire.

The first stage 13 may be positioned such as to be spaced apart from the laser generator 11. The sample 100 may be disposed on the first stage 13. For example, the placing of the sample 100 in the laser treatment apparatus 10 may include placing the sample 100 on the first stage 13 of the laser treatment apparatus 10. The first stage 13 may move in a horizontal direction (e.g., a first direction D1 and a second direction D2) or a vertical direction (e.g., a third direction D3). Therefore, the sample 100 may move in the horizontal direction and the vertical direction.

The reflection mirror 15 may be positioned between the laser generator 11 and the first stage 13. For example, the reflection mirror 15 may be positioned on a movement path of the laser L produced from the laser generator 11. The reflection mirror 15 may reflect the laser L to change the movement path of the laser L.

The lens 17 may be positioned between the reflection mirror 15 and the first stage 13. For example, the lens 17 may be positioned on the movement path of the laser L reflected from the reflection mirror 15. The lens 17 may refract the laser L to focus the laser L on the sample 100 disposed on the first stage 13.

According to an embodiment, a laser may be used in a dicing process in which the wafer WF of FIG. 2 is processed. In this case, the wafer process step (operation S1) and a portion of the sample process step (operation S2) may be performed simultaneously in the laser treatment apparatus 10.

Referring to FIGS. 1, 3, and 4, the sample 100 may have a first surface 100a and a second surface 100b. The first surface 100a and the second surface 100b may be opposite to each other. When the sample 100 is positioned on the first stage 13, the second surface 100b of the sample 100 may be in contact with the first stage 13, and the first surface 100a of the sample 100 may be exposed. For example, the sample 100 may be placed upside down on the first stage 13.

In addition, the sample 100 may include a substrate 120 and a device layer 110 that are in contact with each other. The substrate 120 may have a top surface 121, and the device layer 110 may be positioned on the top surface 121 of the substrate 120. The top surface 121 of the substrate 120 may be parallel to the horizontal direction (e.g., the first direction D1 and the second direction D2). For example, a top surface of the substrate 120 may be the first surface 100a of the sample 100. A top surface of the device layer 110 may be the second surface 100b of the sample 100. The top surface 121 of the substrate 120 may be positioned between the first surface 100a and the second surface 100b of the sample 100.

As the sample 100 is a portion of the wafer WF separated from the wafer WF of FIG. 2, the substrate 120 of the sample 100 may be substantially the same as the substrate of the wafer WF. Moreover, the device layer 110 of the sample 100 may include the semiconductor device of the wafer WF. For example, the substrate 120 of the sample 100 may include monocrystalline silicon, and the top surface 121 of the substrate 120 may have a (100) crystal plane. Furthermore, the third direction D3 orthogonal to the top surface 121 of the substrate 120 may be parallel to a [100] crystal direction.

The laser L produced from the laser generator 11 may be irradiated to the first surface 100a of the sample 100 that is exposed. A focal point, which is formed by the lens 17, of the laser L may be positioned in the sample 100. For example, an energy of the laser L may be concentrated on the focal point to change properties of a substance positioned on the focal point. Thus, a guide line GL may be formed in the sample 100.

A portion of the sample 100 may be removed due to laser ablation to form the guide line GL. For example, a portion of the substrate 120 may be removed to form an opening OP in the substrate 120. For example, the opening OP in the substrate 120 may correspond to the guide line GL. The operation S23 of irradiating the sample 100 with the laser L to form the guide line GL may include forming the opening OP in the substrate 120.

The opening OP may extend from the first surface 100a of the sample 100 toward the second surface 100b of the sample 100 or the top surface 121 of the substrate 120. In addition, the opening OP may extend in the first direction D1. The opening OP may have a width in the second direction D2 that intersects the first direction D1. For example, the opening OP may have a first width W1 at a bottom surface OPb of the opening OP. The first width W1 may range from about 1 μm to about 150 μm. The opening OP may have different widths at a top end and the bottom surface OPb, but embodiments of the present disclosure are not limited thereto.

The bottom surface OPb of the opening OP may be spaced apart in the third direction D3 from the top surface 121 of the substrate 120. In this sense, the opening OP may not completely penetrate the substrate 120. Thus, the opening OP may be spaced apart from the device layer 110. A first thickness T1 may refer to a distance between the bottom surface OPb of the opening OP and the top surface 121 of the substrate 120. The substrate 120 may have a second thickness T2 in the third direction D3. The first thickness T1 may be less than the second thickness T2. For example, the first thickness T1 may be a minimum thickness of the substrate 120, and the second thickness T2 may be a maximum thickness of the substrate 120. The first thickness T1 may range from about 20 um to about 200 um. The second thickness T2 may range from about 100 um to about 1,500 um.

The substrate 120 may include a structural deformation region P1 and a thermal deformation region P2. The structural deformation region P1 and the thermal deformation region P2 may be formed by the laser L. For example, the structural deformation region PI may be an area where a crystal structure of the substrate 120 is altered due to phase change, recrystallization, and/or grain growth which are resulting from direct heating by the laser L. The thermal deformation region P2 may be an area where the substrate 120 has thermal history caused by temperature changes without change of crystal structure.

The structural deformation region P1 and the thermal deformation region P2 may be positioned close to the opening OP. For example, the structural deformation region P1 and the thermal deformation region P2 may extend from the bottom surface OPb of the opening OP onto sidewalls of the opening OP. The structural deformation region P1 may be positioned closer than the thermal deformation region P2 to the opening OP. In this description, the structural deformation region P1 and the thermal deformation region P2 may be integrally called a heat affected zone HAZ.

When physical properties of the laser L satisfy a certain range, the heat affected zone HAZ formed by the laser L may be spaced apart from the device layer 110. For example, the laser L may have a laser power of about 30 W to about 50 W, a laser pulse duration of about 500 fs to about 50 ns, a laser pulse repetition rate of about 100 kHz to about 1,000 kHz, a nominal pulse energy of about 80 μJ to about 200 μJ, a laser wavelength of about 330 nm to about 600 nm, and a laser scanning speed of about 50 mm/s to about 2,000 mm/s.

For example, the laser L according to some embodiments of the present disclosure may have high photon energy per unit area. The laser L may produce multiphoton ionization on the first surface 100a of the sample 100. Since the occurrence of the heat affected zone HAZ is minimized, the heat affected zone HAZ may be spaced apart in the third direction D3 from the device layer 110. Therefore, the semiconductor device included in the device layer 110 may be reduced or prevented from being damaged due to the laser L.

Referring to FIGS. 1, 3, and 5, the formation of the opening OP in the substrate 120 may include sequentially forming a first opening OP1 and a second opening OP2 in the substrate 120. For example, the opening OP may include the first opening OP1 and the second opening OP2.

The first opening OP1 and the second opening OP2 may have different widths from each other. For example, the first opening OP1 may have a second width W2 at a bottom surface thereof. The second opening OP2 may have a third width W3 at a bottom surface thereof. The second width W2 may be greater than the third width W3. The third width W3 may be substantially the same as the first width W1 discussed with reference to FIG. 4. For example, the third width W3 may range from about 1 μm to about 150 μm. The opening OP may have a T-shaped cross-section.

The formation of the first opening OP1 and the second opening OP2 may include allowing (or causing by control by a controller) the first stage 13 to move in the vertical direction (e.g., the third direction D3 or a direction opposite to the third direction D3). For example, the first stage 13 may move in a direction opposite to the third direction D3 to form the first opening OP1. The focal point of the laser L may thus move from the first surface 100a toward the second surface 100b of the sample 100. The migration of the focal point of the laser L may cause a reduction in photon energy per unit area of the laser L and an increase in area of the laser L that removes the substrate 120. As the substrate 120 has an increased area removed by the laser L, there may be a reduction in time required for removing the substrate 120.

Afterwards, the first stage 13 may be allowed to (or caused by control by a controller) move in the third direction D3 to form the second opening OP2. The focal point of the laser L may thus move from the second surface 100b toward the first surface 100a of the sample 100. The migration of the focal point of the laser L may cause an increase in photon energy per unit area of the laser L and a reduction in area of the laser L that removes the substrate 120. The high photon energy per unit area of the laser L may reduce the occurrence of the heat affected zone HAZ. Accordingly, the device layer 110 may be prevented from being damaged due to the laser L.

The heat affected zone HAZ of the substrate 120 may be positioned adjacent to the first opening OP1 and the second opening OP2. A portion of the heat affected zone HAZ adjacent to the first opening OP1 may have a size different from a size of a portion of the heat affected zone HAZ adjacent to the second opening OP2. For example, the photon energy per unit area of the laser L for forming the first opening OP1 may be less than the photon energy per unit area of the laser L for forming the second opening OP2. Therefore, the size of the portion of the heat affected zone HAZ adjacent to the first opening OP1 may be greater than the size of the portion of the heat affected zone HAZ adjacent to the second opening OP2. In this configuration, each of the structural deformation region P1 and the thermal deformation region P2 of the heat affected zone HAZ may become larger in a direction from the second opening OP2 toward the first opening OP1, and may become smaller in a direction from the first opening OP1 toward the second opening OP2.

For example, the substrate 120 may have a plurality of openings that are formed to have their widths different from each other. When a last opening is formed, the laser L may have maximum photon energy per unit area. Thus, the occurrence of the heat affected zone HAZ may be minimized which is adjacent to the device layer 110. Accordingly, the semiconductor device may be reduced or prevented from being damaged.

Referring to FIGS. 1, 6, 7, and 8, the sample destruction step (operation S25) may include placing the guide line GL of the sample 100 on a loading pin 25, using the loading pin 25 to apply a pressure F to the sample 100, and allowing (or causing by control by the controller) a blade 21 to contact the sample 100.

The sample 100 having the guide line GL formed by the laser L may move to a bending test apparatus 20. The bending test apparatus 20 may include a blade 21, a second stage 23, a loading pin 25, and two support pins 27. The bending test apparatus 20 may include a three-point flexural test apparatus, but embodiments of the present disclosure are not limited thereto.

The second stage 23 may be positioned in a lower portion of the bending test apparatus 20. The loading pin 25 may be positioned at a center in the second direction D2 of the second stage 23. In addition, the loading pin 25 may be positioned in the second stage 23. The sample 100 may be positioned on the second stage 23 to allow the first surface 100a of the sample 100 to face toward the second stage 23. For example, the sample 100 may be placed right side up on the second stage 23. The sample 100 may be disposed on the second stage 23 to allow the guide line GL of the sample 100 to vertically overlap the loading pin 25. The guide line GL of the sample 100 may be positioned on the loading pin 25. The loading pin 25 may move in the vertical direction (e.g., the third direction D3).

The blade 21 and the support pins 27 may be positioned on the second surface 100b of the sample 100. The blade 21 may vertically overlap the loading pin 25 and the guide line GL of the sample 100. The blade 21 may move in the vertical direction. For example, the blade 21 may include a diamond knife. The support pins 27 may be adjacent to opposite ends of the sample 100 and in contact with the second surface 100b of the sample 100. The support pins 27 may not vertically overlap the guide line GL of the sample 100. In addition, the support pins 27 may be horizontally spaced apart from the loading pin 25 and the blade 21. For example, the support pins 27 and the blade 21 may be combined into one assembly, but embodiments of the present disclosure are not limited thereto.

The loading pin 25 may move in the third direction D3 to contact the first surface 100a of the sample 100. The loading pin 25 may be in contact with the opening OP of the substrate 120. The loading pin 25 may push the sample 100 in the third direction D3. The loading pin 25 may provide the sample 100 with the pressure F in a direction from the first surface 100a toward the second surface 100b of the sample 100. For example, the pressure F provided from the loading pin 25 may range from about 50 gf to about 150 gf. Thus, the first surface 100a of the sample 100 may be spaced apart in the third direction D3 from the second stage 23. In addition, the first surface 100a and the second surface 100b of the sample 100 may be bent, and the top surface 121 of the substrate 120 (which is a “top” surface with respect to FIG. 7) may also be bent. For example, a compressive stress may be applied to the first surface 100a of the sample 100, and a tensile stress may be applied to the second surface 100b of the sample 100.

In a state where the loading pin 25 provides the sample 100 with the pressure F, the blade 21 may move in a direction opposite to the third direction D3. Therefore, the blade 21 may contact the second surface 100b of the sample 100. As the tensile stress is applied to the second surface 100b of the sample 100, the blade 21 may cause the sample 100 to be easily cut in the third direction D3. For example, the sample destruction step (operation S25) may include cutting the sample 100 in the third direction D3 orthogonal to the top surface 121 of the substrate 120. Therefore, the sample 100 may be divided along the guide line GL, such that a cross-section 100s of the sample 100 may be exposed. The cross-section 100s of the sample 100 may vertically overlap the opening OP and the guide line GL of the sample 100.

According to some embodiments, the substrate 120 may include monocrystalline silicon, and the top surface 121 of the substrate 120 may have the (100) crystal plane. In this case, the sample destruction step (operation S25) may include cutting the sample 100 in the [100] crystal direction orthogonal to the top surface 121 of the substrate 120. Therefore, the sample 100 may be cut in the [100] crystal direction to easily measure the cross-section 100s of the sample 100 in the sample analysis step (operation S3).

However, as monocrystalline silicon has properties of being easily cut in a [100] crystal direction, the sample 100 may be cut in the [100] crystal direction when the first thickness T1 is large. In contrast, the heat affected zone HAZ may cause damage to the semiconductor device of the device layer 110 when the first thickness T1 is small. Therefore, according to embodiments, it the first thickness T1 may have a certain range to easily cut the sample 100 in the [100] crystal direction. For example, when the first thickness T1 is in a range of about 80 μm to about 120 μm, the sample 100 may be easily cut in the crystal direction (or the third direction D3). In this case, no post-treatment process may be separately needed after fracture of the sample 100. Accordingly, it may be possible to reduce cost and time required for processing the sample 100.

Referring to FIGS. 1 and 9, the sample analysis step (operation S3) may include measuring the cross-section 100s of the sample 100 that is cut. For example, scanning electron microscopy (SEM) or transmission electron microscopy (TEM) may be used to measure the cross-section 100s of the sample 100, but embodiments of the present disclosure are not limited thereto.

The sample 100 may include a substrate 120 and a device layer 110 on the substrate 120, and the device layer 110 may include a semiconductor device integrated on the substrate 120. The semiconductor device may include a device isolation layer STI, source/drain patterns SD, word lines, bit lines BL, and data storage patterns DSP. For example, the semiconductor device may be a memory device.

The substrate 120 may be provided therein with the device isolation layer STI that defines an active region. The device isolation layer STI may be positioned in a trench formed by removing a portion of the substrate 120. The device isolation layer STI may be positioned between the source/drain patterns SD. The source/drain patterns SD may be regions where impurities are doped in the substrate 120. For example, the source/drain patterns SD may correspond to a source region and a drain region of the semiconductor device.

Bit-line contacts DC, bit lines BL, and bit-line capping patterns BP may be sequentially provided on some of the source/drain patterns SD. The bit-line contacts DC may electrically connect the bit lines BL to some of the source/drain patterns SD. Bit-line spacers BS may be provided on opposite lateral surfaces of the bit-line contacts DC, the bit lines BL, and the bit-line capping patterns BP. A pair of bit-line spacers BS may cover lateral surfaces of the bit-line contacts DC, the bit lines BL, and bit-line capping patterns BP that vertically overlap with each other.

Storage node contacts BC may be provided between the bit lines BL that are adjacent to each other in the horizontal direction. The storage node contacts BC may be spaced apart from each other in the horizontal direction. The storage node contacts BC may be connected to the source/drain patterns SD that are not coupled to the bit lines BL. Landing pads LP may be provided on the storage node contacts BC. The landing pads LP may be spaced apart from each other in the horizontal direction. The landing pads LP may be connected to corresponding ones of the storage node contacts BC. The landing pads LP may have lower portions that vertically overlap the storage node contacts BC. The landing pads LP may have upper portions that are shifted in the horizontal direction from the lower portions thereof. The upper portions of the landing pads LP may be positioned on the bit-line capping patterns BP. In addition, filling patterns FP may be provided between the landing pads LP that are adjacent to each other in the horizontal direction. The filling patterns FP may surround lateral surfaces of the landing pads LP.

Data storage patterns DSP may be positioned on the landing pads LP. The data storage patterns DSP may be spaced apart from each other in the horizontal direction. The data storage patterns DSP may be connected to corresponding ones of the landing pads LP. The data storage patterns DSP may include bottom electrodes BE, a dielectric layer IL, a top electrode TE, and support patterns SP. The dielectric layer IL may be positioned between the bottom electrodes BE and the top electrode TE, while having a uniform thickness. The support patterns SP may be provided between the bottom electrodes BE that are adjacent to each other in the horizontal direction. Wiring lines CL may be provided on the top electrode TE of the data storage patterns DSP.

For example, the semiconductor device may be a dynamic random access memory (DRAM). However, embodiments of the present disclosure are not limited thereto, and the data storage patterns DSP may include a magnetic tunnel junction pattern. In this case, the semiconductor device may be a magnetic random access memory (MRAM). According to some embodiments, the data storage patterns DSP may include a phase change material or a variable resistance material. In this case, the semiconductor device may be a phase change random access memory (PRAM) or a resistive random access memory (ReRAM).

A sample processing method and a semiconductor device analysis method using the same according to some embodiments of the present disclosure may include irradiating a sample with a laser to form a guide line. The laser may include a femtosecond pulse laser or a nanosecond pulse laser having high photon energy per unit area, and thus a semiconductor device may be prevented from being damaged due to the laser.

In addition, a sample may include a substrate formed of monocrystalline silicon, and a top (or bottom) surface of the substrate may have a (100) crystal plane. The monocrystalline silicon may have properties of being easily cut in a [100] crystal direction. Thus, a distance between the top (or bottom) surface of the substrate and a bottom surface of an opening formed by the laser may be adjusted to facilitate an easy cut of the sample in the [100] crystal direction.

According to embodiments of the present disclosure, the laser treatment apparatus 10 may further include a controller. The controller may be configured to control the laser treatment apparatus 10 to perform its functions. For example, the controller may be configured to control components of the laser treatment apparatus 10 to perform their respective functions, including the semiconductor device analysis method S. For example, the controller may be configured to control operation and/or movement of the laser generator 11, the reflection mirror 15, the lens 17, the first stage 13, and/or at least one actuator connected to the aforementioned components. According to embodiments, the controller may include at least one processor and memory storing computer instructions. The computer instructions, when executed by the at least one processor, may be configured to cause the controller to perform its functions.

Although non-limiting example embodiments of the present disclosure have been described in connection with the accompanying drawings, it will be understood to those skilled in the art that various changes and modifications may be made without departing from the spirit and scope of the present disclosure. It therefore will be understood that the embodiments described above are just illustrative are not limitative in all aspects.

What is claimed is:

1. A sample processing method, comprising:
preparing a sample that has a first surface and a second surface that are opposite to each other;
forming a guide line that extends in a first direction by irradiating the first surface of the sample with a laser; and
destroying the sample along the guide line,
wherein the sample includes a substrate that has a top surface that extends parallel to the first direction,
wherein the top surface of the substrate has a (100) crystal plane, and wherein the laser includes a femtosecond pulse laser or a nanosecond pulse laser.

2. The sample processing method of claim 1, wherein forming the guide line comprises forming an opening that extends from the first surface toward the second surface.

3. The sample processing method of claim 2, wherein a distance between a bottom surface of the opening and the top surface of the substrate is in a range of 80 μm to 120 μm.

4. The sample processing method of claim 2, wherein the opening has a width in a second direction that intersects the first direction, wherein the width of the opening is in a range of 1 μm to 50 μm.

5. The sample processing method of claim 2, wherein the opening includes a first opening and a second opening that overlaps with the first opening,
wherein a width of the first opening is greater than a width of the second opening.

6. The sample processing method of claim 2, wherein the sample further includes a device layer on the top surface of the substrate, and
wherein the opening penetrates a portion of the substrate and is spaced apart from the device layer.

7. The sample processing method of claim 1, wherein the top surface of the substrate is between the first surface and the second surface.

8. The sample processing method of claim 1, wherein the destroying the sample comprises:
placing the guide line of the sample on a loading pin;
applying, by the loading pin, a pressure to the sample; and
contacting the sample with a blade.

9. The sample processing method of claim 1, wherein the destroying the sample comprises cutting the sample in a direction orthogonal to the top surface of the substrate.

10. The sample processing method of claim 9, wherein a cross-section of the sample that is cut vertically overlaps the guide line.

11. A semiconductor device analysis method, comprising:
processing a wafer, that includes a substrate and a device layer on the substrate, such as to obtain a sample from the wafer;
processing the sample obtained from the wafer; and
analyzing the sample,
wherein processing the sample comprises:
forming an opening in the substrate by irradiating a bottom surface of the substrate with a laser; and
destroying the sample along the opening,
wherein a distance between a bottom surface of the opening and a top surface of the substrate is in a range of 80 μm to 120 μm, and
wherein the destroying the sample comprises cutting the sample in a [100] crystal direction.

12. The semiconductor device analysis method of claim 11, wherein the opening penetrates a portion of the substrate and is spaced apart from the device layer.

13. The semiconductor device analysis method of claim 11, wherein the substrate includes monocrystalline silicon, and wherein the top surface of the substrate has a (100) crystal plane.

14. The semiconductor device analysis method of claim 11, wherein the opening extends in a first direction and has a width in a second direction that intersects the first direction, and
wherein the width of the opening is in a range of 1 μm to 50 μm.

15. The semiconductor device analysis method of claim 11, wherein the substrate includes a heat affected zone adjacent to the opening,
wherein the heat affected zone is formed by the laser and is spaced apart from the device layer.

16. The semiconductor device analysis method of claim 11, wherein the device layer includes at least one from among a static random access memory (SRAM), a dynamic random access memory (DRAM), a NAND Flash memory, and a logic circuit.

17. The semiconductor device analysis method of claim 11, wherein the laser has:
a laser power of 30 W to 50 W;
a laser pulse duration of 500 fs to 50 ns;
a laser pulse repetition rate of 100 kHz to 1,000 kHz;
a laser wavelength of 330 nm to 600 nm;
a nominal pulse energy of 80 μJ to 200 μJ; and
a laser scanning speed of 50 mm/s to 2,000 mm/s.

18. The semiconductor device analysis method of claim 11, wherein a thickness of the substrate is in a range of 100 μm to 1,500 μm.

19. The semiconductor device analysis method of claim 11, wherein the analyzing the sample comprising measuring, by a scanning electron microscope (SEM) or a transmission electron microscope (TEM), an exposed cross-section of the sample that is obtained by cutting the sample.

20. The semiconductor device analysis method of claim 11, wherein the opening includes a plurality of openings having different widths from each other.
